(19) United States
(12) Patent Application Publication
(10) Pub. No.: US 2025/0273305 A1
(43) Pub. Date: Aug. 28, 2025

(54) COMPUTER-READABLE RECORDING MEDIUM STORING ALGORITHM SELECTION PROGRAM, ALGORITHM SELECTION METHOD, AND INFORMATION PROCESSING APPARATUS

(71) Applicant: Fujitsu Limited, Kawasaki-shi (JP)

(72) Inventor: Satoshi IMAMURA, Kawasaki (JP)

(73) Assignee: Fujitsu Limited, Kawasaki-shi (JP)

(21) Appl. No.: 18/985,715

(22) Filed: Dec. 18, 2024

(30) Foreign Application Priority Data
Feb. 28, 2024 (JP) 2024-029192

(51) Int. Cl. G16C 20/20

(52) U.S. Cl. CPC G16C 20/20

(57) ABSTRACT

A non-transitory computer-readable recording medium stores an algorithm selection program for causing a computer to execute a process including: in a case where a solution for structure optimization of a molecule is calculated using a quantum chemical calculation algorithm that repeatedly executes calculation for a plurality of cycles, executing a first quantum chemical calculation algorithm for a target molecule and calculating a nuclear gradient norm for each cycle; executing the first quantum chemical calculation algorithm for a cycle in which the nuclear gradient norm is equal to or more than a first threshold; and executing a second quantum chemical calculation algorithm of which execution time is longer than the first quantum chemical calculation algorithm for a cycle in which the nuclear gradient norm is less than the first threshold.

### CROSS-REFERENCE TO RELATED APPLICATION

This application is based upon and claims the benefit of priority of the prior Japanese Patent Application No. 2024-29192, filed on Feb. 28, 2024, the entire contents of which are incorporated herein by reference.

### FIELD

The embodiment discussed herein is related to a computer-readable recording medium storing an algorithm selection program and the like.

### BACKGROUND

As a method for analyzing the structure and properties of a molecule from an electronic state, there is quantum chemical calculation. Quantum chemical calculation is based on calculating the energy of a target molecule. By obtaining the energy of a molecule, various characteristics may be grasped, which may be used for drug discovery and development of new materials.

International Publication Pamphlet No. WO 2011/036952 is disclosed as related art.

### SUMMARY

According to an aspect of the embodiments, a non-transitory computer-readable recording medium stores an algorithm selection program for causing a computer to execute a process including: in a case where a solution for structure optimization of a molecule is calculated using a quantum chemical calculation algorithm that repeatedly executes calculation for a plurality of cycles, executing a first quantum chemical calculation algorithm for a target molecule and calculating a nuclear gradient norm for each cycle; executing the first quantum chemical calculation algorithm for a cycle in which the nuclear gradient norm is equal to or more than a first threshold; and executing a second quantum chemical calculation algorithm of which execution time is longer than the first quantum chemical calculation algorithm for a cycle in which the nuclear gradient norm is less than the first threshold.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for describing a technical problem related to algorithm switching;

FIG. 2 is a diagram illustrating the calculation precision of a nuclear gradient norm of lightweight algorithms;

FIG. 3A is a diagram for describing processing of an information processing apparatus according to the present embodiment;

FIG. 3B is a diagram illustrating the number of cycles taken for structure optimization by each algorithm;

FIG. 4 is a functional block diagram illustrating a configuration of the information processing apparatus according to the present embodiment;

FIG. 5 is a diagram illustrating an example of a data structure of algorithm candidate data;

FIG. 6 is a diagram illustrating an example of a data structure of an algorithm list;

FIG. 7 is a flowchart illustrating a processing procedure of the information processing apparatus according to the present embodiment;

FIG. 8 is a flowchart illustrating a processing procedure of algorithm list creation processing;

FIG. 9 is a flowchart illustrating a processing procedure of structure optimization processing;

FIG. 10 is a diagram illustrating an example of a hardware configuration of a computer that implements functions similar to those of the information processing apparatus of the present embodiment;

FIG. 11 is a diagram for describing molecular structure optimization;

FIG. 12 is a diagram illustrating the relationship between energy difference and nuclear gradient norm over the course of cycles;

FIG. 13 is a diagram for describing the relationship between precision level and calculation amount of quantum chemical calculation; and

FIG. 14 is a diagram for describing the relationship between execution time and precision related to structure optimization.

### DESCRIPTION OF EMBODIMENTS

Representative processing performed in quantum chemical calculation includes structure optimization of molecules. In structure optimization, a stable structure of a molecule is obtained by molecular structure calculation. For example, in structure optimization, energy calculation and nuclear gradient calculation are repeatedly executed while changing the structure of a molecule, and when the energy difference or nuclear gradient norm falls below a threshold, convergence occurs.

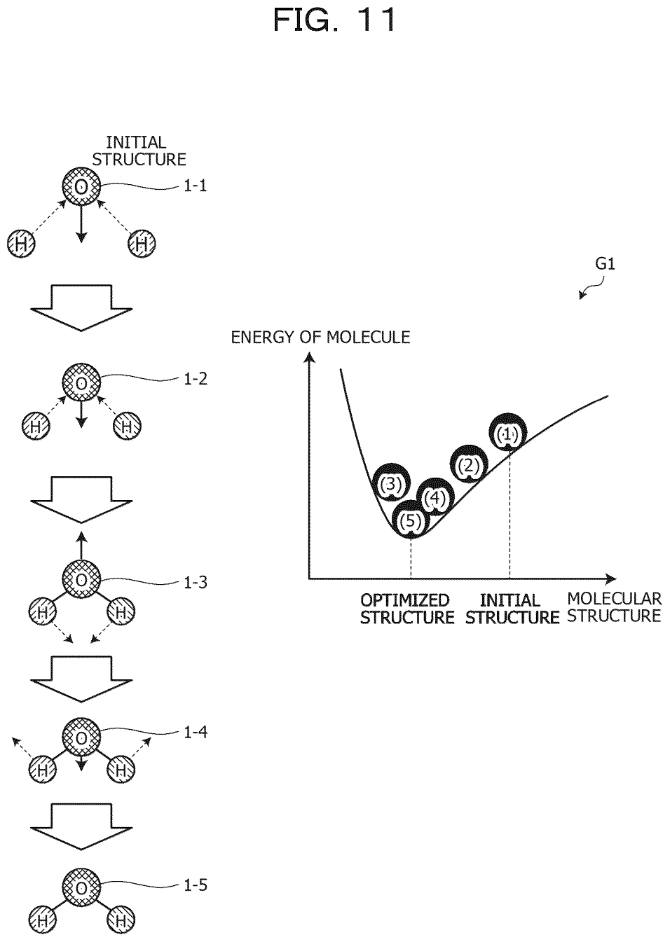

FIG. 11 is a diagram for describing molecular structure optimization. In FIG. 11, description is given using the water molecule “H2O” as an example. Graph G1 indicates the energy of molecule in each structure of the molecule (water molecule). The horizontal axis of graph G1 is an axis corresponding to molecular structure, and the vertical axis is an axis corresponding to energy of molecule. Hereinafter, energy of molecule will be referred to as “energy” as appropriate.

In structure optimization, the position of each atom is displaced little by little according to the vector of the force acting on each atom, and the structure is optimized little by little. For example, the initial structure of the molecule is molecular structure 1-1. The position in graph G1 corresponding to molecular structure 1-1 and the energy is (1). When molecular structure 1-1 is changed to molecular structure 1-2 by structure optimization, the position in graph G1 corresponding to the molecular structure and energy is (2).

Subsequently, when molecular structure 1-2 is changed to molecular structure 1-3 by structure optimization, the position in graph G1 corresponding to the molecular structure and energy is (3). When molecular structure 1-3 is changed to molecular structure 1-4, the position in graph G1 corresponding to the molecular structure and energy is (4). When molecular structure 1-4 is changed to molecular structure 1-5, the position in graph G1 corresponding to the molecular structure and energy is (5).

Molecular structure 1-5 has the minimum value of energy, and is the optimized structure.

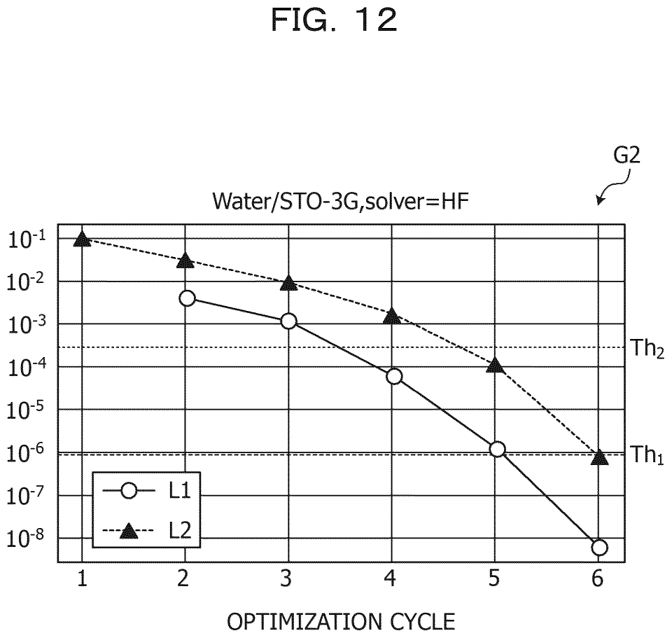

FIG. 12 is a diagram illustrating the relationship between energy difference and nuclear gradient norm over the course of cycles. The horizontal axis of graph G2 is an axis corresponding to cycle (optimization cycle), and the vertical axis corresponds to value (value of energy difference or value of nuclear gradient norm). Line L1 indicates the relationship between cycle and energy difference. Line L2 indicates the relationship between cycle and nuclear gradient norm.

For example, the processing from update of a molecular structure to calculation of energy and nuclear gradient of the updated molecular structure is one cycle. Hereinafter, the n-th cycle is referred to as cycle n. An energy difference indicates a difference between the energy of a molecular structure in the previous cycle and the energy of the molecular structure in the current cycle. A nuclear gradient norm is the length of a sum of vectors acting on each atom obtained from a calculation result of nuclear gradient. An energy difference may be calculated in two or more number of cycles. A nuclear gradient norm may be calculated in one or more number of cycles.

In graph G2 illustrated in FIG. 12, in cycle “6”, the energy difference is less than a threshold Th₁, and the nuclear gradient norm is less than a threshold Th₂. For example, in cycle “6”, the molecule has the optimized structure, and it is indicated that the number of cycles for reaching the optimized structure (the number of optimization cycles) is “6”.

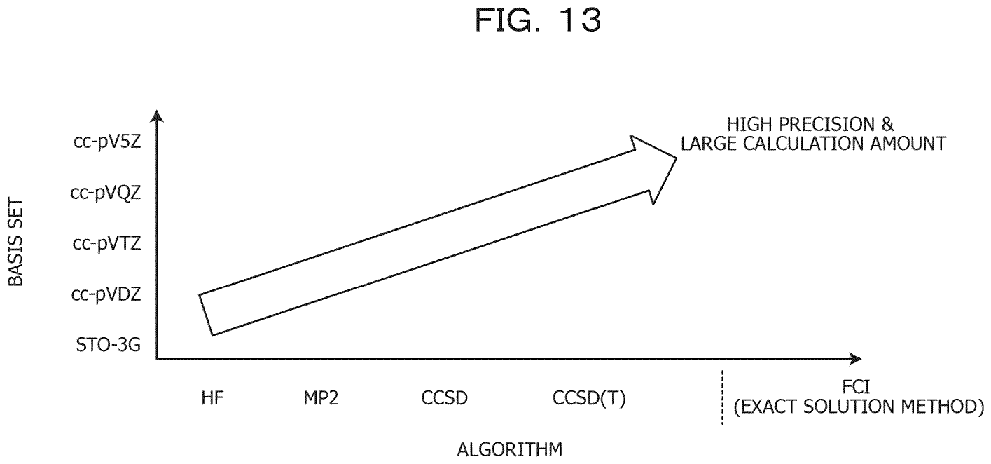

Next, the relationship between precision level and calculation amount of the quantum chemical calculation described above will be described. FIG. 13 is a diagram for describing the relationship between precision level and calculation amount of quantum chemical calculation. As illustrated in FIG. 13, the precision level and calculation amount are determined according to the pair of basis set and algorithm.

A basis set is a set of functions used for describing the state of electrons in a molecule. For example, basis sets include STO-3G, cc-pVDZ, cc-pVTZ, cc-pVQZ, cc-pV5Z, and the like. The problem scale for handling molecules increases in the order of STO-3G, cc-pVDZ, cc-pVTZ, cc-pVQZ, and cc-pV5Z (STO-3G has the smallest problem scale).

An algorithm is a technique for analyzing the electronic structure of a molecule, and energy and nuclear gradient are obtained. An algorithm performs analysis using any of the basis functions included in a basis set. Algorithms include hartree fock method (HF), second-order moller-plesset method (MP2), coupled cluster method (CC method), and full configuration interaction method (FCI). CCSD is one of the CC methods, and SD is an abbreviation for “singles and doubles”. CCSD(T) is one of the CC methods, and SD(T) is an abbreviation for “singles, doubles, and perturbative triples”. The calculation amount increases and the precision basically increases in the order of HF, MP2, CCSD, CCSD(T), and FCI.

For example, when quantum chemical calculation is executed by a pair of a basis set with a large problem scale and an algorithm with a large calculation amount, the precision level is increased, but the execution time is increased. On the other hand, when quantum chemical calculation is executed by a pair of a basis set with a small problem scale and an algorithm with a small calculation amount, the precision level is lowered, but the execution time is shortened.

In the above-described related art, there is a trade-off relationship between execution time and precision related to structure optimization.

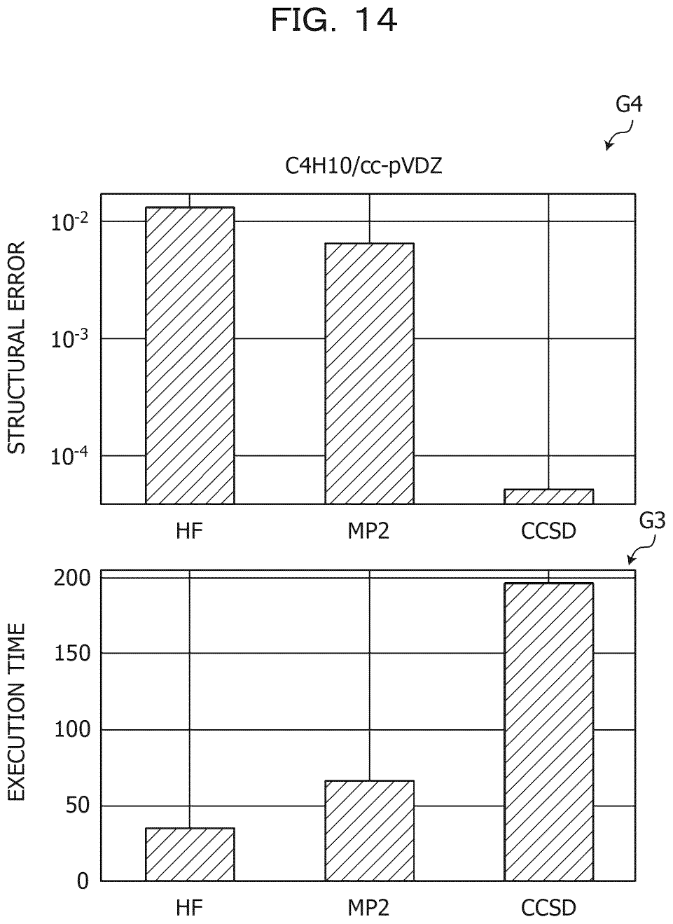

FIG. 14 is a diagram for describing the relationship between execution time and precision related to structure optimization. In FIG. 14, as an example, the target molecule for structure optimization is “C4H10”, and the algorithms are HF, MP2, and CCSD. The basis set used by each algorithm is “cc-pVDZ”.

Graph G3 illustrates the execution time for each algorithm. The vertical axis of graph G3 is an axis corresponding to execution time, and the horizontal axis is an axis corresponding to algorithm. As illustrated in graph G3, the execution time increases in the order of HF, MP2, and CCSD.

Graph G4 illustrates the structural error (precision) for each algorithm. The larger the structural error, the lower the precision. The vertical axis of graph G4 is an axis corresponding to structural error, and the horizontal axis is an axis corresponding to algorithm. As illustrated in graph G4, the structural error decreases in the order of HF, MP2, and CCSD.

As illustrated in graphs G3 and G4, it may be seen that an algorithm with a large calculation amount has to be used for high-precision structure optimization, and an algorithm with a larger calculation amount takes a longer time for structure optimization. The execution time is calculated by “execution time=(energy calculation time+nuclear gradient calculation time)×number of cycles”, and the energy calculation time and nuclear gradient calculation time increase to the power of the molecular scale.

For example, it is desired to shorten the time taken for structure optimization.

In one aspect, an object of the present disclosure is to provide an algorithm selection program, an algorithm selection method, and an information processing apparatus capable of shortening the time taken for structure optimization.

Hereinafter, an embodiment of an algorithm selection program, an algorithm selection method, and an information processing apparatus disclosed in the present application will be described in detail with reference to the drawings. This disclosure is not limited by this embodiment.

### Embodiment

As an approach for shortening the execution time of high-precision structure optimization, it is conceivable to use a lightweight algorithm in the first several cycles and switch to a high-precision algorithm in the optimal cycle. For example, there is an approach in which an initial structure of a molecule is brought close to an optimized structure first by using HF, MP2, or the like, and then the optimized structure is calculated by CCSD or the like.

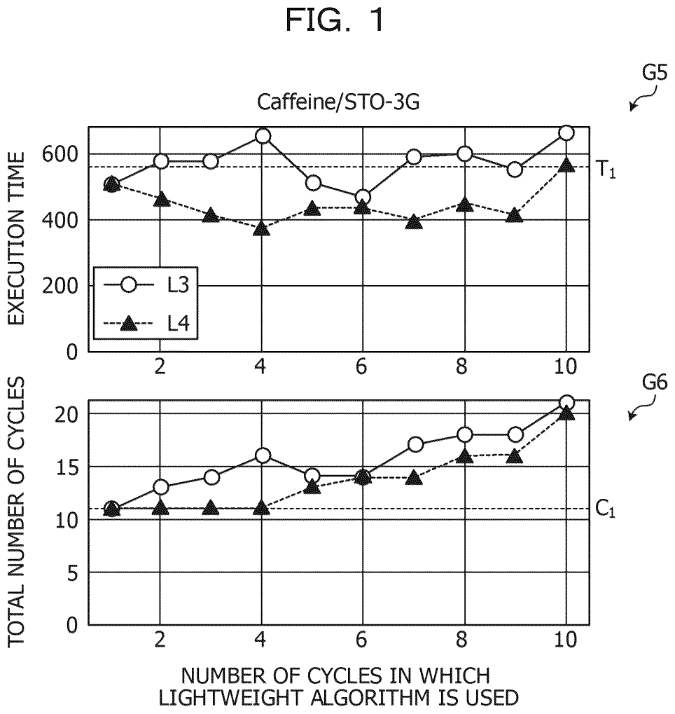

A technical problem in a case of switching from a lightweight algorithm to a high-precision algorithm will be described. FIG. 1 is a diagram for describing a technical problem related to algorithm switching. For example, the lightweight algorithms are HF and MP2, and the high-precision algorithm is CCSD. The basis set used by each algorithm is “STO-3G”, and the target molecule for structure optimization is “Caffeine”.

First, graph G5 will be described. Graph G5 illustrates the execution time of structure optimization in a case where the lightweight algorithm is executed for x cycles (the number of cycles x) first, and then the algorithm is switched to the high-precision algorithm. This execution time is the total execution time of the execution time of the lightweight algorithm and the execution time of the high-precision algorithm. The horizontal axis of graph G5 corresponds to number of cycles in which the lightweight algorithm is executed, and the vertical axis is an axis corresponding to execution time. T₁ is the execution time of structure optimization in a case where only the high-precision algorithm is used from the beginning.

Line L3 in graph G5 indicates the execution time in a case where CCSD is executed after HF is executed first for x cycles. Line L4 in graph G5 indicates the execution time in a case where CCSD is executed after MP2 is executed first for x cycles.

In graph G5, the number of cycles x for which the execution time of line L3 is equal to or more than T₁ indicates that the execution time in the case where CCSD is executed after HF is executed first for x cycles is longer than the execution time in a case where CCSD is executed from the beginning. In graph G5, the number of cycles x for which the execution time of line L4 is equal to or more than T₁ indicates that the execution time in the case where CCSD is executed after MP2 is executed first for x cycles is longer than the execution time in the case where CCSD is executed from the beginning.

For example, when attention is focused on the number of cycles “x=4” in graph G5, the execution time of line L3 is the execution time equal to or more than T₁. This indicates that the execution time in the case where CCSD is executed after HF is executed first for four cycles is longer than the execution time in the case where CCSD is executed from the beginning. On the other hand, the execution time of line L4 is the execution time of less than T₁. This indicates that the execution time in the case where CCSD is executed after MP2 is executed first for four cycles is shorter than the execution time in the case where CCSD is executed from the beginning.

Next, graph G6 will be described. Graph G6 illustrates the total number of cycles of structure optimization in the case where the lightweight algorithm is executed for x cycles (the number of cycles x) first, and then the algorithm is switched to the high-precision algorithm. This total number of cycles is the total number of cycles of the number of cycles of the lightweight algorithm and the number of cycles of the high-precision algorithm. The horizontal axis of graph G6 corresponds to number of cycles in which the lightweight algorithm is executed, and the vertical axis is an axis corresponding to total number of cycles. C₁ is the total number of cycles in the case where only the high-precision algorithm is used.

Line L3 in graph G6 indicates the total number of cycles in the case where CCSD is executed after HF is executed first for x cycles. Line L4 in graph G6 indicates the execution time in the case where CCSD is executed after MP2 is executed first for x cycles.

In graph G6, the number of cycles x for which the total number of cycles of line L3 is equal to or more than C₁ indicates that the total number of cycles in the case where CCSD is executed after HF is executed first for x cycles is larger than the total number of cycles in the case where CCSD is executed from the beginning. In graph G6, the number of cycles x for which the total number of cycles of line L4 is equal to or more than C₁ indicates that the total number of cycles in the case where CCSD is executed after MP2 is executed first for x cycles is larger than the total number of cycles in the case where CCSD is executed from the beginning.

For example, when attention is focused on the number of cycles “x=4” in graph G6, the total number of cycles of line L3 is the total number of cycles equal to or more than C₁. This indicates that the total number of cycles in the case where CCSD is executed after HF is executed first for four cycles is larger than the total number of cycles in the case where CCSD is executed from the beginning. On the other hand, the total number of cycles of line L4 is substantially the same as C₁. This indicates that the total number of cycles in the case where CCSD is executed after MP2 is executed first for four cycles is substantially the same as the total number of cycles in the case where CCSD is executed from the beginning.

As described in graphs G5 and G6 of FIG. 1, if the number of cycles in which the lightweight algorithm is used is too large, the total number of cycles increases and it is difficult to shorten the execution time. If the number of cycles in which the lightweight algorithm is used is too large, the precision may deteriorate. The point at which the total number of cycles begins to increase differs depending on the type of lightweight algorithm. Therefore, it is important to determine the number of cycles in which the lightweight algorithm is used so that the execution time taken for structure optimization is minimized.

The technical problem in the case of switching from a lightweight algorithm to a high-precision algorithm has been described above.

As described in graph G2 of FIG. 12, a nuclear gradient norm may be calculated from the first cycle. Attention is focused on the fact that since the nuclear gradient norm is about 10⁻² to 10⁻¹ in the first several cycles regardless of the algorithm, precise calculation may be performed even with a lightweight algorithm.

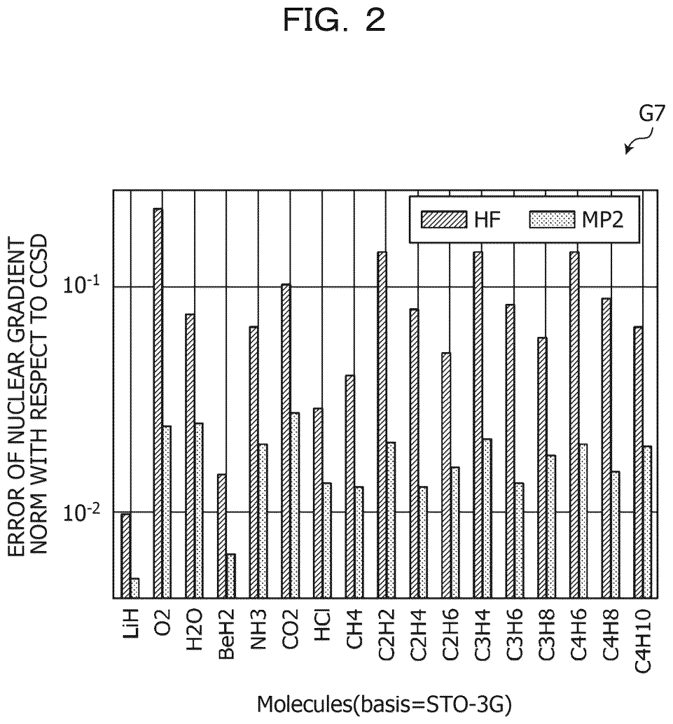

FIG. 2 is a diagram illustrating the calculation precision of a nuclear gradient norm of lightweight algorithms. Graph G7 in FIG. 2 illustrates errors with respect to CCSD in a case where nuclear gradient norm calculation is performed using lightweight algorithms (HF and MP2) for each molecule. The horizontal axis of graph G7 is an axis corresponding to types of molecules, and the vertical axis is an axis corresponding to error (error of nuclear gradient norm with respect to CCSD; hereinafter, simply referred to as error in the description of FIG. 2).

In the example illustrated in FIG. 2, the molecules used for structure optimization are LiH, O2, H2O, BeH2, NH3, CO2, HCl, CH4, C2H2, C2H4, C2H6, C3H4, C3H6, C3H8, C4H6, C4H8, and C4H10.

For example, in a case where HF is used as the algorithm, the maximum error among the errors for the respective molecules is “0.2”. In a case where MP2 is used as the algorithm, the maximum error among the errors for the respective molecules is “0.03”. As long as the nuclear gradient norm is larger than these maximum errors, calculation may be executed precisely even when a lightweight algorithm is used.

For example, in a case where structure optimization is performed, calculation may be executed precisely even when HF is used in the cycle during which the nuclear gradient norm is “0.2” or more. Similarly, in the case where structure optimization is performed, calculation may be executed precisely even when MP2 is used in the cycle during which the nuclear gradient norm is “0.03” or more.

The information processing apparatus according to the present embodiment focuses on the maximum error of nuclear gradient norm described in FIG. 2, and switches the algorithm in the process of executing structure optimization, thereby making it possible to shorten the execution time of high-precision structure optimization. In the following description, the information processing apparatus according to the present embodiment is referred to as an “information processing apparatus 100”.

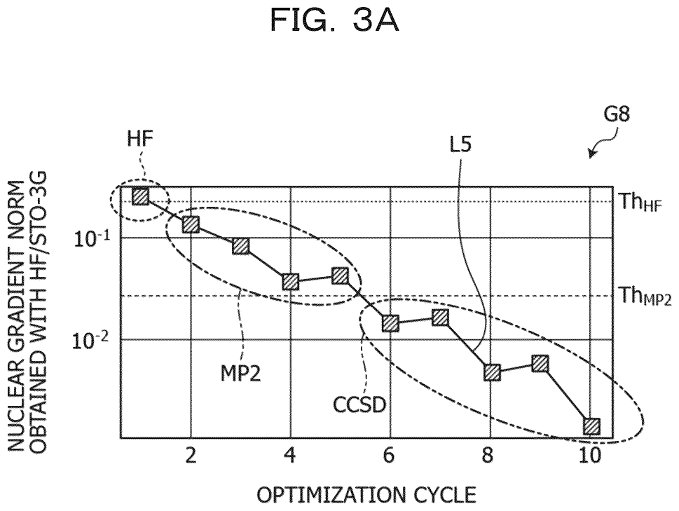

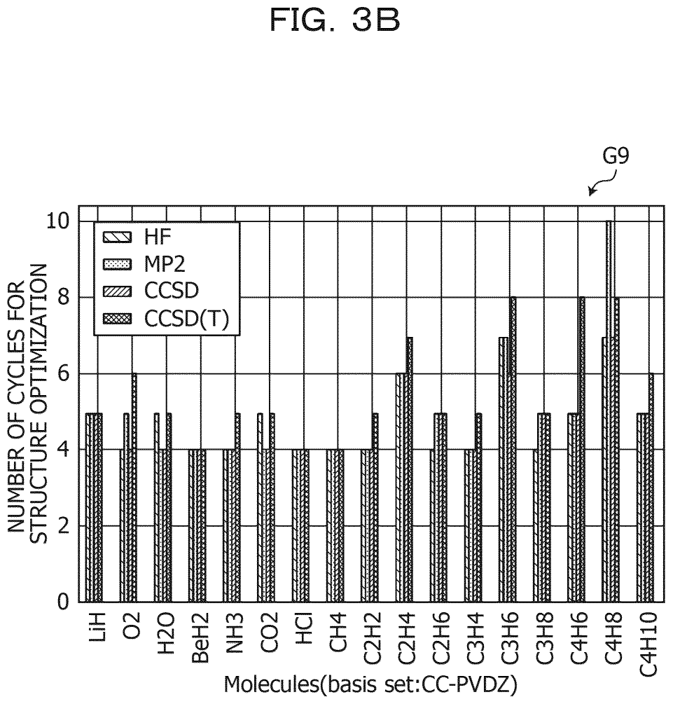

FIG. 3A is a diagram for describing processing of the information processing apparatus according to the present embodiment. In the present embodiment, a case where the algorithms HF, MP2, and CCSD are switched stepwise will be described. The basis set used by each algorithm is “STO-3G”, and the target molecule for structure optimization is “Caffeine”. The information processing apparatus 100 performs actual execution processing after pre-execution processing is performed.

The pre-execution processing executed by the information processing apparatus 100 will be described. The information processing apparatus 100 selects the algorithm “HF” and the basis set “STO-3G”, and executes structure optimization for the target molecule. This specifies the nuclear gradient norm for each cycle, which is calculated with the algorithm “HF” and the basis set “STO-3G”. For example, the nuclear gradient norm for each cycle is illustrated by line L5 in graph G8. The pair of algorithm and basis set is denoted as “HF/STO-3G”.

The vertical axis of graph G8 is an axis corresponding to nuclear gradient norm obtained with HF/STO-3G, and the horizontal axis is an axis corresponding to the number of cycles in which HF/STO-3G is executed. The example illustrated in FIG. 3A indicates that the optimized structure is specified with HF/STO-3G during pre-execution, with the number of cycles “10”.

Threshold Th_{HF} illustrated in graph G8 is the maximum error of HF described in FIG. 2. Threshold Th_{MP2} illustrated in graph G8 is the maximum error of MP2 described in FIG. 2.

The pre-execution processing executed by the information processing apparatus 100 has been described above.

The actual execution processing executed by the information processing apparatus 100 will be described. The information processing apparatus 100 selects an algorithm to be used for each cycle based on the result of the pre-execution processing (line L5 in graph G8) and threshold Th_{HF} and threshold Th_{MP2}.

The information processing apparatus 100 selects the algorithm “HF” in the cycle period in which the nuclear gradient norm obtained with HF/STO-3G is equal to or more than threshold ThHF. The information processing apparatus 100 selects the algorithm “MP2” in the cycle period in which the nuclear gradient norm obtained with HF/STO-3G is less than threshold Th_{HF} and equal to or more than threshold Th_{MP2}. The information processing apparatus 100 selects the algorithm “CCSD” in the cycle period in which the nuclear gradient norm obtained with HF/STO-3G is less than threshold Th_{MP2}. The basis set to be used is designated by a user (it may be other than STO-3G).

In the example illustrated in FIG. 3A, the information processing apparatus 100 selects “HF” in the first cycle, selects “MP2” in the second to fifth cycles, and selects “CCSD” in the sixth and subsequent cycles.

The information processing apparatus 100 executes structure optimization for the molecule based on the selected result. For example, the information processing apparatus 100 executes energy calculation and nuclear gradient calculation by “HF” in the first cycle. The information processing apparatus 100 executes energy calculation and nuclear gradient calculation by “MP2” in the second to fifth cycles. The information processing apparatus 100 executes energy calculation and nuclear gradient calculation by “CCSD” in the sixth and subsequent cycles.

As described above, the information processing apparatus 100 according to the present embodiment selects an algorithm to be used for each cycle based on the result of the pre-execution processing (line L5 in graph G8) and threshold Th_{HF} and threshold Th_{MP2}, and executes structure optimization based on the selection result. As a result, a lightweight algorithm may be selected in a cycle period in which precise calculation may be performed even with a lightweight algorithm, and thereafter, a high-precision algorithm may be selected. For example, the time taken for structure optimization may be shortened while maintaining precision.

FIG. 3B is a diagram illustrating the number of cycles taken for structure optimization by each algorithm. In FIG. 3B, as an example, the target algorithms are HF, MP2, CCSD, and CCSD(T). The horizontal axis of graph G9 in FIG. 3B is an axis corresponding to type of molecule, and the vertical axis is an axis corresponding to the number of cycles for structure optimization. The number of cycles for structure optimization is the number of cycles taken for completion of structure optimization.

As illustrated in FIG. 3B, it may be seen that the number of cycles for structure optimization of other algorithms (MP2, CCSD, and CCSD(T)) is not significantly different from the number of cycles for structure optimization of HF. From this, as described in FIG. 3A, it may be said that even if HF is replaced with another algorithm, the number of cycles for structure optimization does not change greatly.

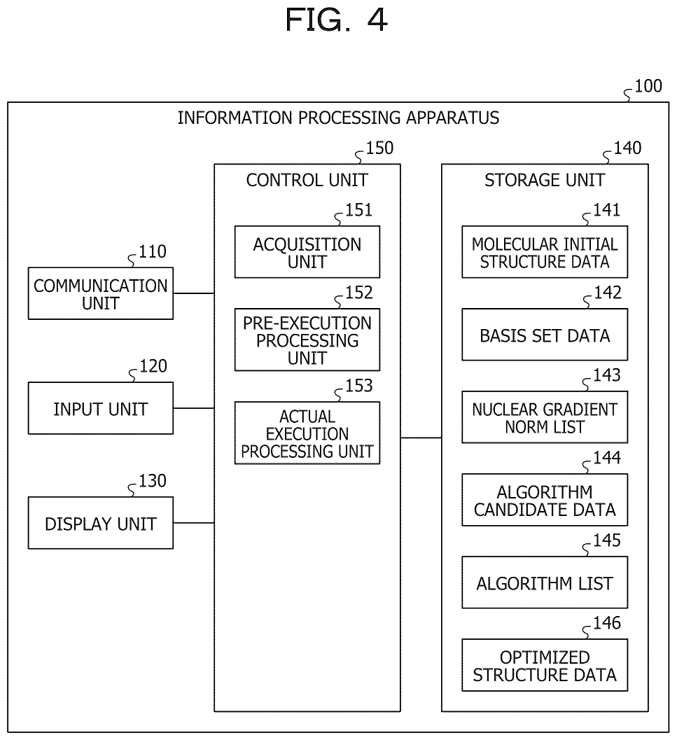

Next, a configuration example of the information processing apparatus 100 of the present embodiment will be described. FIG. 4 is a functional block diagram illustrating a configuration of the information processing apparatus according to the present embodiment. As illustrated in FIG. 4, the information processing apparatus 100 includes a communication unit 110, an input unit 120, a display unit 130, a storage unit 140, and a control unit 150.

The communication unit 110 executes data communication with an external device or the like via a network. The communication unit 110 is a network interface card (NIC) or the like. For example, the communication unit 110 may acquire, from the external device or the like, molecular initial structure data 141, basis set data 142, algorithm candidate data 144, and the like.

The input unit 120 is an input device that inputs various types of information to the control unit 150 of the information processing apparatus 100. For example, the input unit 120 corresponds to a keyboard, a mouse, a touch panel, or the like.

The display unit 130 is a display device that displays information output from the control unit 150.

The storage unit 140 includes the molecular initial structure data 141, the basis set data 142, a nuclear gradient norm list 143, the algorithm candidate data 144, an algorithm list 145, and optimized structure data 146. The storage unit 140 is a memory or the like.

The molecular initial structure data 141 is data of an initial structure of a target molecule of structure optimization.

The basis set data 142 includes basis sets such as STO-3G, cc-pVDZ, cc-pVTZ, cc-pVQZ, and cc-pV5Z. The basis set used in structure optimization is designated in advance by a user.

The nuclear gradient norm list 143 is a list generated by the pre-execution processing, and the nuclear gradient norm calculated in each cycle is set therein. For example, the nuclear gradient norm list 143 is information corresponding to line L5 in graph G8 described in FIG. 3A.

In the algorithm candidate data 144, algorithms to be used in a case where structure optimization is performed for a molecule are set. For example, in the algorithm candidate data 144, types of algorithms and thresholds are associated with each other.

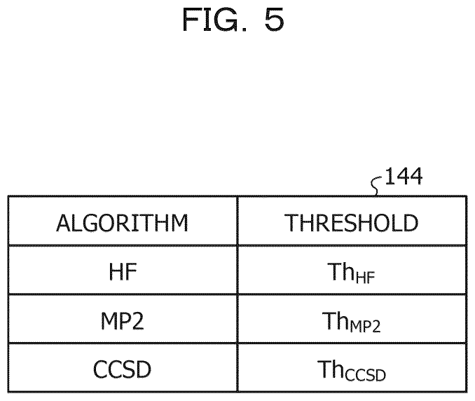

FIG. 5 is a diagram illustrating an example of a data structure of algorithm candidate data. As illustrated in FIG. 5, in the algorithm candidate data 144, algorithms and thresholds are associated with each other. As the algorithms, the types of algorithms described above are set. A threshold is the maximum error corresponding to each algorithm. Description of the maximum error is similar to the description given in FIG. 3A. For example, the order of the elements (records) of the algorithm candidate data is descending order of thresholds from the top.

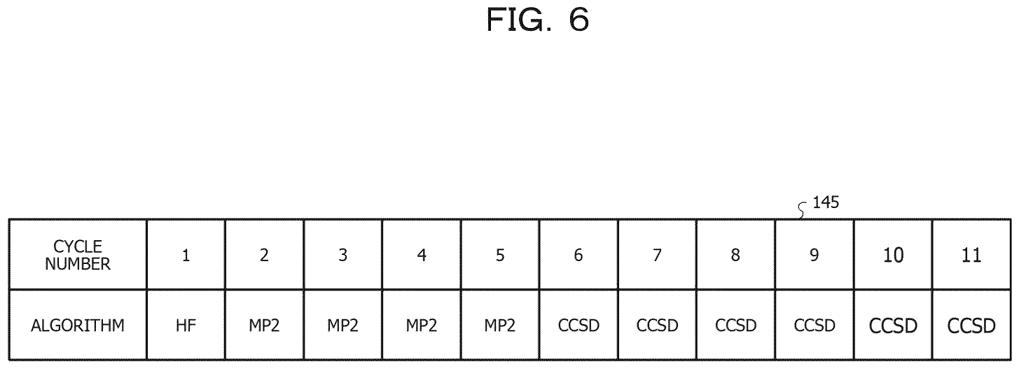

In the algorithm list 145, an algorithm used for each cycle is set. FIG. 6 is a diagram illustrating an example of a data structure of the algorithm list. In the example illustrated in FIG. 6, a cycle number and the algorithm used in the corresponding cycle are associated with each other.

The optimized structure data 146 is an execution result of structure optimization for the molecular initial structure data 141.

Next, description proceeds to that of the control unit 150. The control unit 150 includes an acquisition unit 151, a pre-execution processing unit 152, and an actual execution processing unit 153. The control unit 150 is a central processing unit (CPU), a graphics processing unit (GPU), or the like.

The acquisition unit 151 acquires the molecular initial structure data 141, the basis set data 142, the algorithm candidate data 144, and the like via the communication unit 110. The acquisition unit 151 may acquire the molecular initial structure data 141, the basis set data 142, the algorithm candidate data 144, and the like from the input unit 120.

The acquisition unit 151 stores the acquired molecular initial structure data 141, basis set data 142, algorithm candidate data 144, and the like in the storage unit 140.

The pre-execution processing unit 152 performs the pre-execution processing described in FIG. 3A. For example, the pre-execution processing unit 152 executes structure optimization for the molecular initial structure data 141 with “HF/STO-3G”.

The pre-execution processing unit 152 calculates energy and nuclear gradient and calculates a nuclear gradient norm for each cycle in the process of structure optimization. The pre-execution processing unit 152 registers the relationship between the number of cycles and the nuclear gradient norm in the nuclear gradient norm list 143.

Other description related to the pre-execution processing unit 152 is similar to the description of the pre-execution processing described in FIG. 3A.

The actual execution processing unit 153 performs the actual execution processing described in FIG. 3A. For example, the actual execution processing unit 153 creates the algorithm list 145 by selecting an algorithm to be used for each cycle based on the nuclear gradient norm list 143 and the algorithm candidate data 144. The basis set used in the actual execution processing is designated in advance by a user.

The actual execution processing unit 153 compares the nuclear gradient norm of each cycle in the nuclear gradient norm list 143 with the threshold in the algorithm candidate data 144. The actual execution processing unit 153 selects the algorithm “HF” in a cycle period in which the nuclear gradient norm is equal to or more than threshold Th_{HF} (for example, the first cycle).

The actual execution processing unit 153 selects the algorithm “MP2” in a cycle period in which the nuclear gradient norm is less than threshold Th_{HF} and equal to or more than threshold Th_{MP2} (for example, the second cycle to the fifth cycle). The actual execution processing unit 153 selects the algorithm “CCSD” in a cycle period in which the nuclear gradient norm is less than threshold Th_{MP2} (for example, the sixth cycle to the eleventh cycle).

By the actual execution processing unit 153 executing the above processing, the algorithm list 145 illustrated in FIG. 6 is created.

The actual execution processing unit 153 selects an algorithm for each number of cycles based on the created algorithm list 145, and executes structure optimization for the molecular initial structure data 141. For example, in structure optimization, the actual execution processing unit 153 selects and executes the algorithm “HF” in the first cycle. The actual execution processing unit 153 selects and executes the algorithm “MP2” in the second cycle to the fifth cycle. The actual execution processing unit 153 selects and executes the algorithm “CCSD” in the sixth cycle to the eleventh cycle.

The actual execution processing unit 153 stores the execution result of structure optimization in the storage unit 140 as the optimized structure data 146. The actual execution processing unit 153 may output the optimized structure data 146 to the display unit 130 for display.

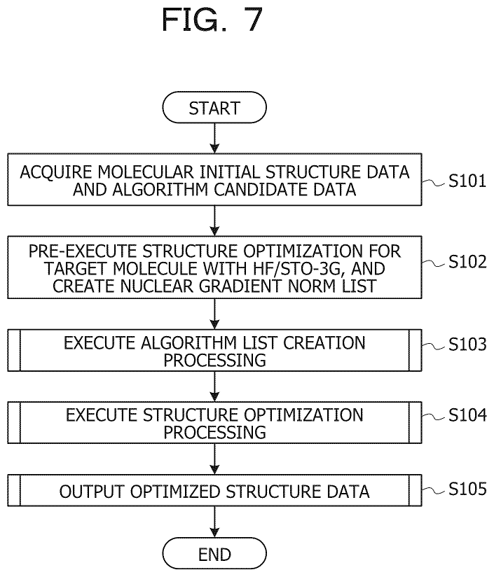

Next, an example of a processing procedure of the information processing apparatus 100 according to the present embodiment will be described. FIG. 7 is a flowchart illustrating a processing procedure of the information processing apparatus according to the present embodiment. The acquisition unit 151 of the information processing apparatus 100 acquires the molecular initial structure data 141 and the algorithm candidate data 144 (step S101).

The pre-execution processing unit 152 of the information processing apparatus 100 pre-executes structure optimization for a target molecule with HF/STO-3G, and creates the nuclear gradient norm list 143 (step S102).

The actual execution processing unit 153 of the information processing apparatus 100 executes algorithm list creation processing (step S103). The actual execution processing unit 153 executes structure optimization processing (step S104).

The actual execution processing unit 153 outputs the optimized structure data 146 (step S105).

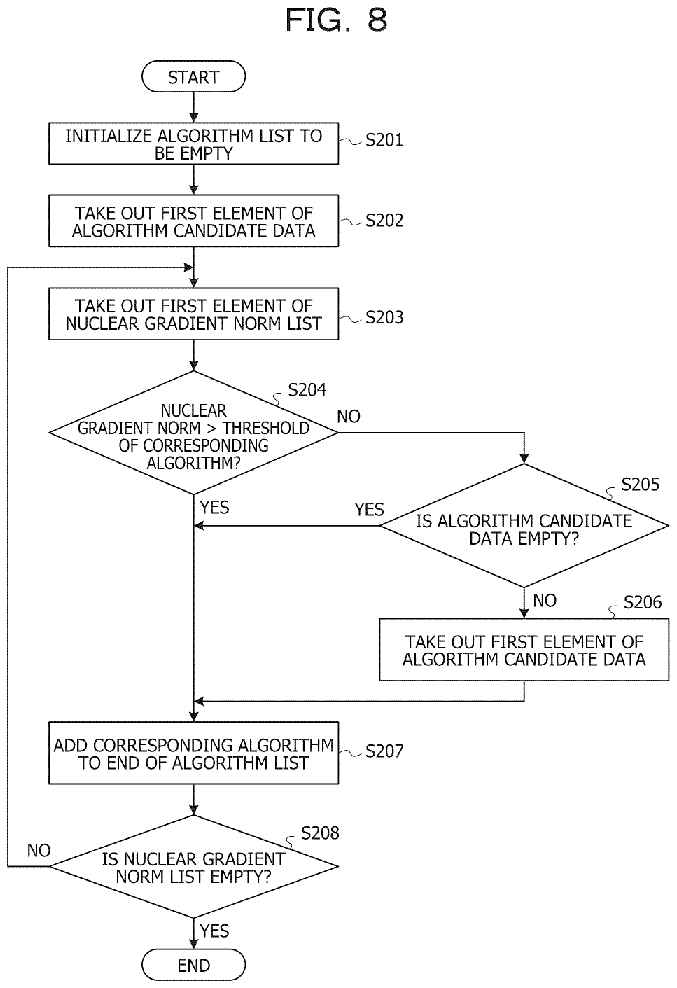

Next, an example of a processing procedure of the algorithm list creation processing described in step S103 of FIG. 7 will be described. FIG. 8 is a flowchart illustrating a processing procedure of the algorithm list creation processing. As illustrated in FIG. 8, the actual execution processing unit 153 of the information processing apparatus 100 initializes the algorithm list 145 to be empty (step S201).

The actual execution processing unit 153 takes out the first element of the algorithm candidate data 144 (step S202). The actual execution processing unit 153 takes out the first element of the nuclear gradient norm list 143 (step S203).

When the nuclear gradient norm is larger than the threshold of the corresponding algorithm (step S204, Yes), the actual execution processing unit 153 proceeds to step S207. On the other hand, when the nuclear gradient norm is not larger than the threshold of the corresponding algorithm (step S204, No), the actual execution processing unit 153 proceeds to step S205.

When the algorithm candidate data 144 is empty (step S205, Yes), the actual execution processing unit 153 proceeds to step S207. On the other hand, when the algorithm candidate data 144 is not empty (step S205, No), the actual execution processing unit 153 takes out the first element of the algorithm candidate data 144 (step S206), and proceeds to step S207.

The actual execution processing unit 153 adds the corresponding algorithm to the end of the algorithm list 145 (step S207). When the nuclear gradient norm list is not empty (step S208, No), the actual execution processing unit 153 proceeds to step S203. On the other hand, when the nuclear gradient norm list is empty (step S208, Yes), the actual execution processing unit 153 ends the algorithm list creation processing.

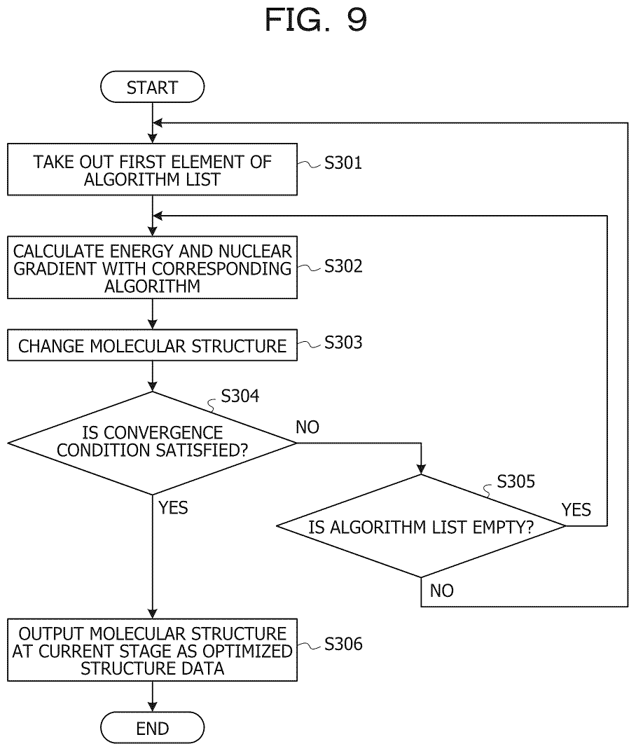

Next, an example of a processing procedure of the structure optimization processing described in step S104 of FIG. 7 will be described. FIG. 9 is a flowchart illustrating a processing procedure of the structure optimization processing. As illustrated in FIG. 9, the actual execution processing unit 153 of the information processing apparatus 100 takes out the first element of the algorithm list 145 (step S301).

The actual execution processing unit 153 calculates energy and nuclear gradient with the corresponding algorithm (step S302). The actual execution processing unit 153 changes the molecular structure (step S303).

When a convergence condition is not satisfied (step S304, No), the actual execution processing unit 153 proceeds to step S305. When the algorithm list 145 is empty (step S305, Yes), the actual execution processing unit 153 proceeds to step S302. On the other hand, when the algorithm list 145 is not empty (step S305, No), the actual execution processing unit 153 proceeds to step S301.

When the convergence condition is satisfied (step S304, Yes), the actual execution processing unit 153 outputs the molecular structure at the current stage as the optimized structure data 146 (step S306).

Next, the effects of the information processing apparatus 100 according to the present embodiment will be described. The information processing apparatus 100 selects an algorithm to be used for each cycle based on a result of pre-execution processing and a threshold set for each algorithm, and executes structure optimization based on the selection result. As a result, a lightweight algorithm may be selected in a cycle period in which precise calculation may be performed even with a lightweight algorithm, and thereafter, a high-precision algorithm may be selected. For example, the time taken for structure optimization may be shortened while maintaining precision.

The information processing apparatus 100 creates an algorithm list in which an algorithm to be used for each cycle is selected based on a result of pre-execution processing and a threshold set for each algorithm. The information processing apparatus 100 may execute structure optimization by switching from a lightweight algorithm to a high-precision algorithm in a stepwise manner in accordance with the algorithm list.

In the present embodiment, description has been given with the basis set used in each algorithm in the pre-execution processing as “STO-3G”, but the present disclosure is not limited thereto, and a different basis set may be used for each algorithm. However, it is desirable that a basis set of a small scale is selected in order to shorten the execution time of pre-execution processing.

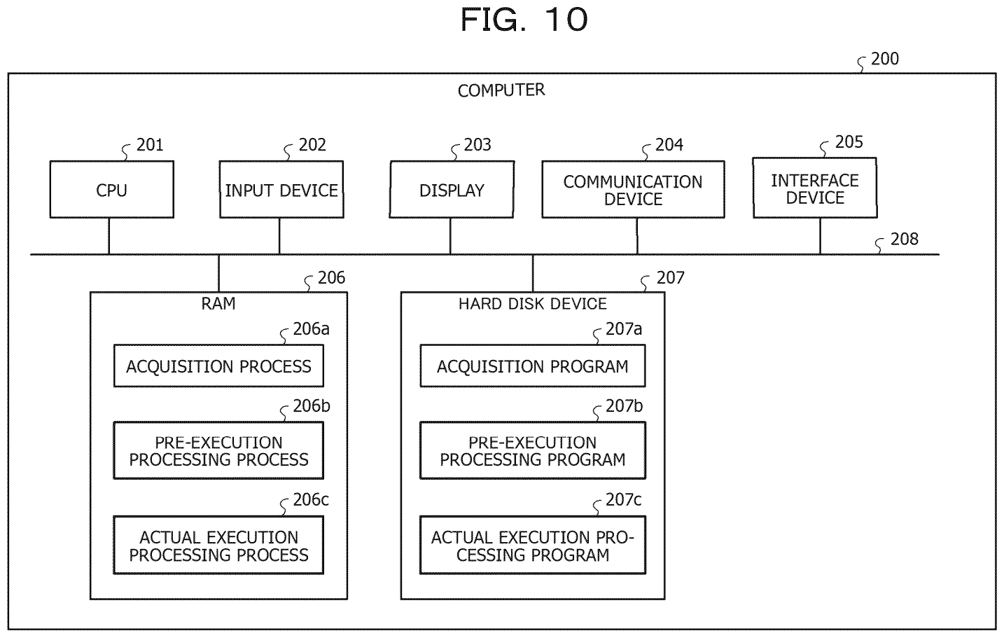

Next, an example of a hardware configuration of a computer that implements functions similar to those of the information processing apparatus 100 described above will be described. FIG. 10 is a diagram illustrating an example of a hardware configuration of a computer that implements functions similar to those of the information processing apparatus of the present embodiment.

As illustrated in FIG. 10, a computer 200 includes a CPU 201 that executes various types of arithmetic processing, an input device 202 that receives input of data from a user, and a display 203. The computer 200 includes a communication device 204 that exchanges data with an external device or the like via a wired or wireless network, and an interface device 205. The computer 200 includes a random-access memory (RAM) 206 that temporarily stores various types of information, and a hard disk device 207. The devices 201 to 207 are coupled to a bus 208.

The hard disk device 207 includes an acquisition program 207a, a pre-execution processing program 207b, and an actual execution processing program 207c. The CPU 201 reads the programs 207a to 207c and loads them to the RAM 206.

The acquisition program 207a functions as an acquisition process 206a. The pre-execution processing program 207b functions as a pre-execution processing process 206b. The actual execution processing program 207c functions as an actual execution processing process 206c.

The processing of the acquisition process 206a corresponds to the processing of the acquisition unit 151. The processing of the pre-execution processing process 206b corresponds to the processing of the pre-execution processing unit 152. The processing of the actual execution processing process 206c corresponds to the processing of the actual execution processing unit 153.

The programs 207a to 207c do not have be stored in the hard disk device 207 from the beginning. For example, each program is stored in a “portable physical medium”, such as a flexible disk (FD), a compact disc read-only memory (CD-ROM), a Digital Versatile Disc (DVD), a magneto-optical disk, or an integrated circuit (IC) card, to be inserted into the computer 200. The computer 200 may read and execute the programs 207a to 207c.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an algorithm selection program for causing a computer to execute a process comprising:
in a case where a solution for structure optimization of a molecule is calculated using a quantum chemical calculation algorithm that repeatedly executes calculation for a plurality of cycles, executing a first quantum chemical calculation algorithm for a target molecule and calculating a nuclear gradient norm for each cycle;
executing the first quantum chemical calculation algorithm for a cycle in which the nuclear gradient norm is equal to or more than a first threshold; and
executing a second quantum chemical calculation algorithm of which execution time is longer than the first quantum chemical calculation algorithm for a cycle in which the nuclear gradient norm is less than the first threshold.

2. The non-transitory computer-readable recording medium according to claim 1, wherein the computer is further caused to execute a process of executing the second quantum chemical calculation algorithm for a cycle in which the nuclear gradient norm is less than the first threshold and equal to or more than a second threshold, and executing a third quantum chemical calculation algorithm of which execution time is longer than the second quantum chemical calculation algorithm for a cycle in which the nuclear gradient norm is less than the second threshold.

3. The non-transitory computer-readable recording medium according to claim 1, wherein the second quantum chemical calculation algorithm has higher precision than the first quantum chemical calculation algorithm.

4. The non-transitory computer-readable recording medium according to claim 2, wherein the third quantum chemical calculation algorithm has higher precision than the second quantum chemical calculation algorithm.

5. The non-transitory computer-readable recording medium according to claim 2, wherein the computer is further caused to execute a process of selecting a quantum chemical calculation algorithm to be executed for the each cycle from the first quantum chemical calculation algorithm, the second quantum chemical calculation algorithm, and the third quantum chemical calculation algorithm based on the nuclear gradient norm for each cycle, the first threshold, the second threshold, and the third threshold.

6. An algorithm selection method for causing a computer to execute a process comprising:
in a case where a solution for structure optimization of a molecule is calculated using a quantum chemical calculation algorithm that repeatedly executes calculation for a plurality of cycles, executing a first quantum chemical calculation algorithm for a target molecule and calculating a nuclear gradient norm for each cycle;
executing the first quantum chemical calculation algorithm for a cycle in which the nuclear gradient norm is equal to or more than a first threshold; and
executing a second quantum chemical calculation algorithm of which execution time is longer than the first quantum chemical calculation algorithm for a cycle in which the nuclear gradient norm is less than the first threshold.

7. The algorithm selection method according to claim 6, wherein the computer is further caused to execute a process of executing the second quantum chemical calculation algorithm for a cycle in which the nuclear gradient norm is less than the first threshold and equal to or more than a second threshold, and executing a third quantum chemical calculation algorithm of which execution time is longer than the second quantum chemical calculation algorithm for a cycle in which the nuclear gradient norm is less than the second threshold.

8. The algorithm selection method according to claim 6, wherein the second quantum chemical calculation algorithm has higher precision than the first quantum chemical calculation algorithm.

9. The algorithm selection method according to claim 7, wherein the third quantum chemical calculation algorithm has higher precision than the second quantum chemical calculation algorithm.

10. The algorithm selection method according to claim 7, wherein the computer is further caused to execute a process of selecting a quantum chemical calculation algorithm to be executed for the each cycle from the first quantum chemical calculation algorithm, the second quantum chemical calculation algorithm, and the third quantum chemical calculation algorithm based on the nuclear gradient norm for each cycle, the first threshold, the second threshold, and the third threshold.

11. An information processing apparatus comprising:
a memory; and
a processor coupled to the memory and configured to:
in a case where a solution for structure optimization of a molecule is calculated using a quantum chemical calculation algorithm that repeatedly executes calculation for a plurality of cycles, execute a first quantum chemical calculation algorithm for a target molecule and calculate a nuclear gradient norm for each cycle;
execute the first quantum chemical calculation algorithm for a cycle in which the nuclear gradient norm is equal to or more than a first threshold; and
execute a second quantum chemical calculation algorithm of which execution time is longer than the first quantum chemical calculation algorithm for a cycle in which the nuclear gradient norm is less than the first threshold.

12. The information processing apparatus according to claim 11, wherein to the processor executes the second quantum chemical calculation algorithm for a cycle in which the nuclear gradient norm is less than the first threshold and equal to or more than a second threshold, and executes a third quantum chemical calculation algorithm of which execution time is longer than the second quantum chemical calculation algorithm for a cycle in which the nuclear gradient norm is less than the second threshold.

13. The information processing apparatus according to claim 11, wherein the second quantum chemical calculation algorithm has higher precision than the first quantum chemical calculation algorithm.

14. The information processing apparatus according to claim 12, wherein the third quantum chemical calculation algorithm has higher precision than the second quantum chemical calculation algorithm.

15. The information processing apparatus according to claim 12, wherein the processor selects a quantum chemical calculation algorithm to be executed for the each cycle from the first quantum chemical calculation algorithm, the second quantum chemical calculation algorithm, and the third quantum chemical calculation algorithm based on the nuclear gradient norm for each cycle, the first threshold, the second threshold, and the third threshold.
